(19) United States
(12) Patent Application Publication
(10) Pub. No.: US 2020/0260843 A1
(43) Pub. Date: Aug. 20, 2020

(54) Heartware LVAD/RVAD Fashionable medical bag device cover

(71) Applicant: Lisa Harmon Long, Lexington, SC (US)

(72) Inventor: Lisa Harmon Long, Lexington, SC (US)

(21) Appl. No.: 16/501,083

(22) Filed: Feb. 20, 2019

(51) Int. Cl. A45F 3/02; A45F 3/12; A45C 3/06

(52) U.S. Cl. CPC A45F 3/02; A61M 1/122; A45C 3/06; A45F 3/12

(57) ABSTRACT

HeartWare medical device bag cover kit comprises: (a) a three dimensional, generally rectangular-shape cover with a closing flap; a cover means a thing which lies around something, especially in order to protect or conceal it; right and left side opening/slit for driveline accessibility; (b) at least 3 magnetic fasteners; (c) a detachable cover for the carrying strap, which will be adjustable in length, therefore coincided with the existing medical grade strap, that is provided for the device; (d) a separate shoulder pad will also be included with a thicker padding and coordinating fabric(s).

All covering will be sold as a kit for the Lvad/Rvad device bag, including the adjustable strap cover that will give patients the freedom to determine the cosmetic appearance, without altering the function and safety of the device, as provided by the manufacturer.

### RELATED APPLICATIONS

This application refers to U. S. Provisional Application Ser. No. 16/501,083 filed for Medical Bag Cover (Fashionable) (Note: Inventor changed the name to HeartWare LVAD/RVAD Fashionable Medical Bag Device Cover to be more descriptive) on Jun. 18, 2018 which is included herein in its entirety by reference.

### BACKGROUND OF THE INVENTION

### Field of the Invention

The present invention is a medical device cover for use by patients with Left Ventricular Assist Devices (LVAD) and/or Right Ventricular Assist Devices (RVAD) which are surgically implanted in a person's body.

### Background

LVADs and RVADs, also known as VADs, are life-sustaining medical devices surgically implanted within the body to assist with cardiac function for patients diagnosed with late stage heart failure.

The LVAD and RVAD have both internal and external components. The internal component is the pump that attaches to the heart, and the external components include two exterior batteries, a controller (which monitors the heart), and a driveline. A driveline cable, which is connected to the VAD internally, exits through the skin in the abdomen and connects to the external hardware (i.e. the controller and battery supply). The external components of both devices must always be close to the body.

LVAD and/or RVAD patients have limited options for carrying their external hardware and being able to function normally in a public setting. Currently, a support bag is needed for the safety of the batteries, controller, and partially exposed driveline. The messenger-style bag provided with the VAD equipment was intentionally designed using the highest quality materials by the makers of the HeartWare LVAD/RVAD to ensure the protection of the external driveline and the batteries and controller within the bag.

There is a present need for cosmetic accessories that will cover the provided HeartWare LVAD and RVAD bags in order to camouflage the exterior of the provided bag. This invention will provide patients with the freedom to determine the cosmetic appearance of their LVAD/RVAD devices without altering the function of the device(s) as provided by the manufacturer.

### BRIEF SUMMARY OF THE INVENTION

The present invention addresses the aforementioned concerns; by providing an option for LVAD/RVAD patients that are searching for a more fashionable solution to carrying their provided external hardware bag. The cosmetic cover will be offered in many different colors, materials/fabrics, styles and can be accessorized with;“but not limited to”; additional embellishments such as pockets, zippers, buttons, handle(s) and tassels to add personalized detail and keep bags up to date with the current fashion trends

This fashionable cover will help patients conceal the exterior components of their LVAD/RVAD and create the illusion that the patient is carrying a purse or messenger-style bag. The cover(s) will be practical and functional for both men and women. The cover(s) will help patients regain a sense of normalcy when in public.

The cover(s) integrates with the existing HeartWare external hardware bag; which will result in an unaltered functionality and maintains the integrity and safety of the medical device as provided from the manufacturer. The patient will insert their HeartWare medical device bag inside the cosmetic cover; in order to conceal the medical device. The bag cover; will also provide a thicker shoulder strap pad to help distribute the weight of the medical bag and its contents.

The bag cover, strap cover and extra padded shoulder pad, will be sold as a kit in order to assimilate all pieces together for the simplicity of use.

### BRIEF DESCRIPTION OF THE DRAWINGS

Numerous objects, features and related advantages of the present invention will become more fully appreciated as the same becomes better understood; when considered in conjunction with the accompanying drawings:

FIG. 1 is a front plan view of the cosmetic cover that is adapted to camouflage the existing LVAD and RVAD medical device bags;

FIG. 2 is an exploded perspective view of the cosmetic bag cover and the medical device bag. The illustration will give a clearer understanding of how the bag assimilates prior to placement into the external cover;

FIG. 3 is a back-plan view of the bag cover with belt loops for the safety belt that is recommended by the manufacturer;

FIG. 4 is a front plan view of the cover with flap opened. This will show the magnetic closure of the underneath portion of the frontal flap and front panel cover, where the flap and bag will be joined together. Also, shown is the right and left tab, with right tab opened to give a clearer visual of the exterior male and female magnetic closures;

FIG. 5 is a view of the strap cover that was adapted to camouflage the medical device safety strap; a clearer understanding shown in the view and how the strap of the medical device bag, integrates through the medical device cover;

FIG. 6 is a view of the strap cover, with its attaching strap connector (Lobster Clasp) that will integrate all pieces of the kit together;

FIG. 7 is a view of the actual prototype with a partial view of the front panel with its side opening/slit;

FIG. 8 refers to both right and left side (s) panel(s) of the medical device bag cover;

FIG. 9 is a view of the tab and the front panel of the medical device bag cover;

FIG. 10 is a view of the bottom of medical device bag cover; with attached front and back panel(s).

FIG. 11 is a view of the actual prototype medical device bag and strap cover.

### LIST OF REFERENCE NUMBERS USED IN DESCRIPTIONS

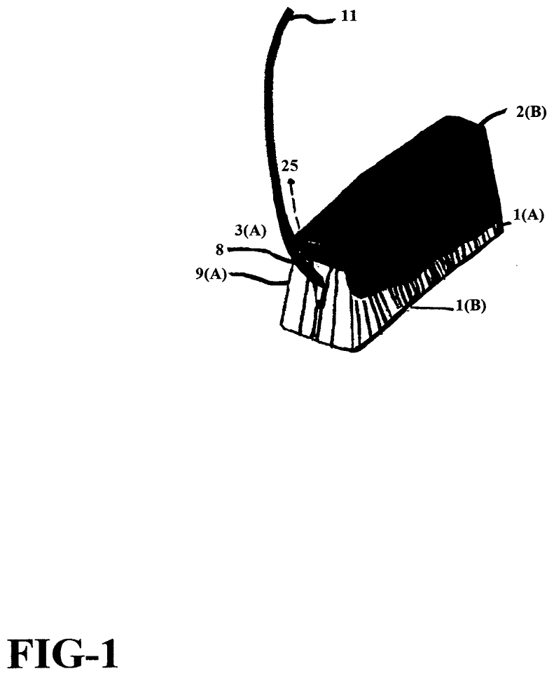

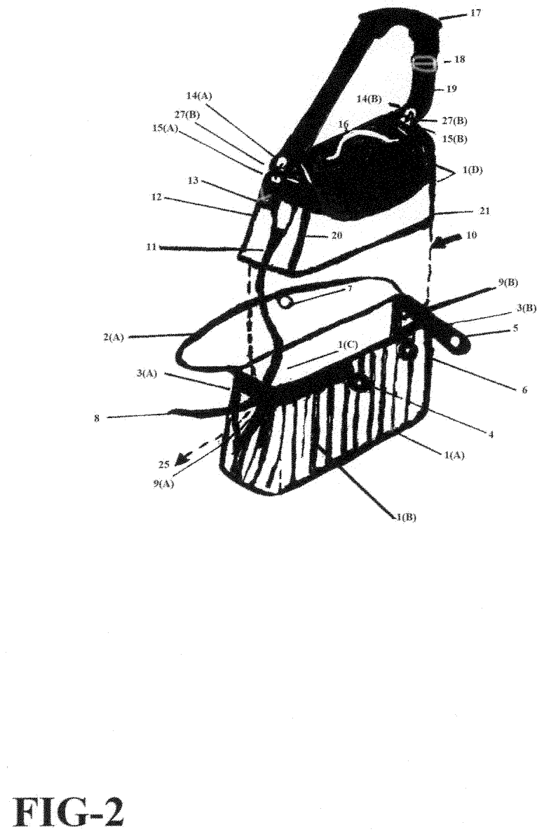

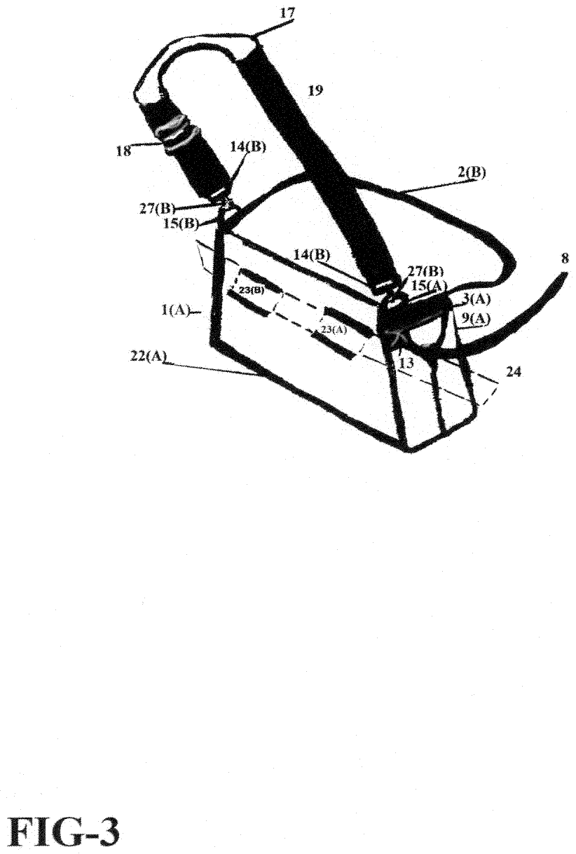

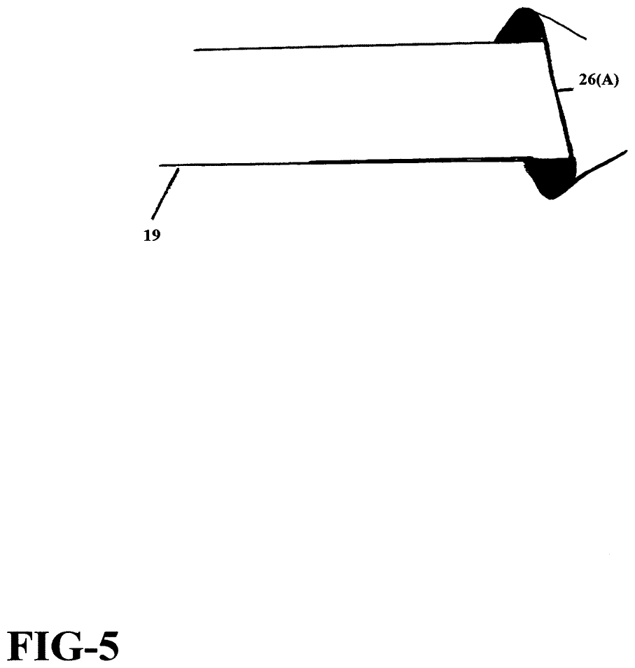

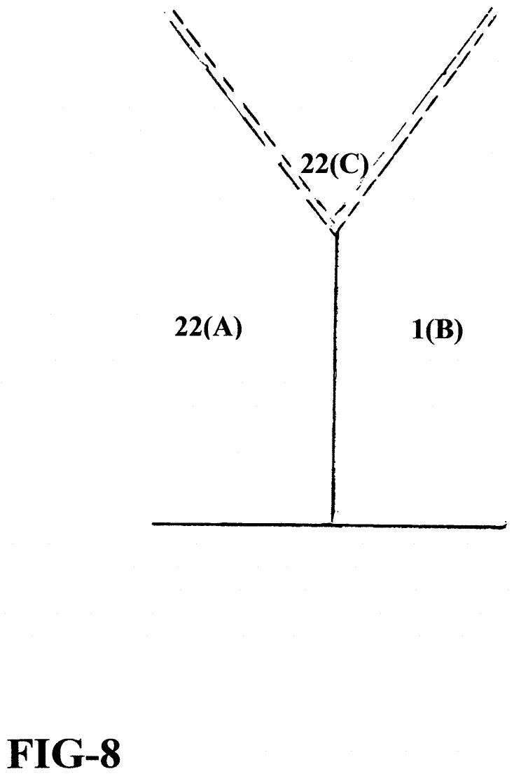

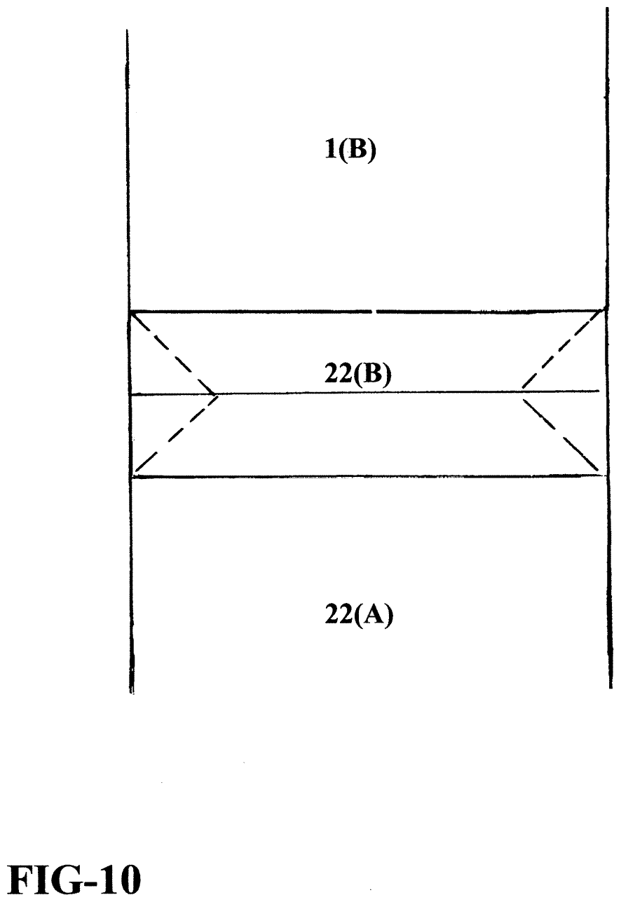

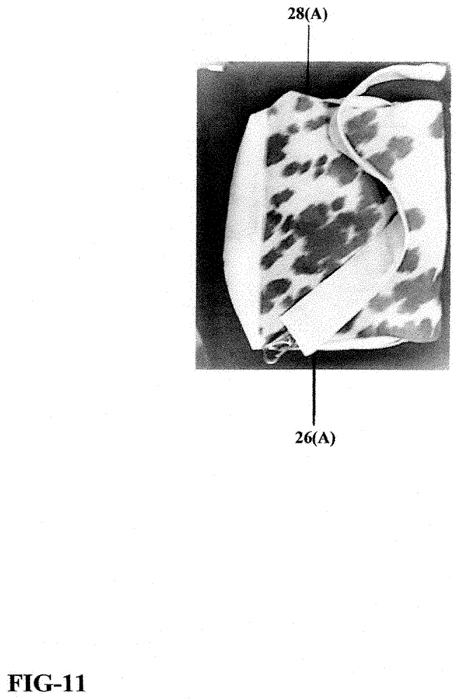

**Medical device bag cover refers to; a cover made for camouflaging the peripheral support device bag
**Medical device bag refers to; a bag containing the peripheral support devices for (LVAD/RVAD)
1.(A) medical device bag cover FIGS. 1,2,3,4
1.(B) front panel of the medical bag cover FIGS. 1,2,4,9,10
1.(C) internal fabric lining for medical device bag cover FIGS. 2,4
1.(D) medical device bag FIG. 2
2.(A) interior flap of medical device bag cover FIGS. 2,4
2.B) exterior flap of medical device bag cover FIGS. 1,3,
3.(A) right tabs FIGS. 1,2,3,4,9
3.(B) left tabs FIGS. 2,4
4.) male magnetic portion of the closure for middle of front panel of the medical device bag cover FIGS. 2,4
5.) female magnetic portion of closure for tabs FIGS. 2,4
6.) male magnetic portion of tabs FIGS. 2,4
7.) female magnetic portion of interior flap closure FIGS. 2,4
8.) partial internal driveline as it looks exiting through opening FIGS. 1,2,3,4
9.(A) right side panel opening/slit view for driveline exit from interior of the medical device cover FIGS. 1,2,3,4,7
9.(B) left side panel opening/slit view for driveline exit from interior of the medical device cover FIGS. 1,2,3,4
10.) refers to how the two bags will integrate together FIG. 2
11.) driveline FIGS. 1,2,3
12.) right side opening/slit for driveline exit from medical device bag FIG. 2
13.) refers to the fabric attached to medical device bags D rings 15(a) and 15 (b) for securing hardware in place FIGS. 2,3
14.(A) right side rectangle D ring attachment for the medical device bag strap FIGS. 2,3
14.(B) left side rectangle D ring attachment for the medical device strap FIGS. 2,3
15.(A) right D ring attachment for the medical device bag strap FIGS. 2,3
15.(B) left side D ring attachment for the medical device strap FIGS. 2,3
16.) refers to the handle on the medical device bag FIG. 2
17.) medical device bags removeable shoulder pad FIGS. 2,3
18.) medical device bags adjustable strap buckle FIGS. 2,3
19.) medical device bag strap FIGS. 2,3
20.(A) medical device bag flap FIG. 2
21.) front panel of medical device bag FIG. 3
22.(A) back panel of medical device cover FIGS. 3,8,10
22.(B) refers to the bottom of the medical device bag cover that will be created from 1(b) front panel of the medical bag cover and 22(a.) back panel of the medical device cover FIG. 10
22.(C) refers to both the right and left (side (s)of medical device bag cover, that is created by 1 (b) and 22 (a)FIG. 8
23.(A) right back panel belt loop for medical device cover FIG. 3
23.(B) left back panel belt loop for medical device cover FIG. 3
24.). waist safety belt, as it would look during its merger through the belt loops FIG. 3
25.) referring to the direction that the driveline would be negotiated through the interior of the medical device bag cover and exiting through the opening/lit of 9(a) and under tab of 3(a) FIG. 2,4
26.(A) refers to the medical device bag strap cover FIGS. 5,6
27.(A) lobster clasps FIGS. 2,3,6
28.(A) refers to the actual prototype bag, and shoulder strap cover 26(a) used to cover for the medical device bag and safety strap FIG. 11
28.(B) refers to a removable shoulder pad (not shown in drawings and is just for reference purposes only) that will be completed by adding extra padding and will be sewn in coordinating fabrics to be combined with the 1(a)medical device bag cover and 26 (a) medical device bag strap cover

### DETAILED DESCRIPTION OF THE INVENTION

In the following description, like reference characters designate like or corresponding parts throughout the several views. Also, in the following description, it is to be understood that such terms as “front,” “back,” “side (s), “bottom, “opening/slits, and like words of convenience and or not to be construed as limiting terms. Referring in more detail to the drawing, the invention will now be described.

The present invention addresses the aforementioned concerns by providing an option for LVAD/RVAD patients that are searching for a more fashionable solution to carrying their provided external hardware bag. LVADs and or RVADs, also known as VADs, is a life sustaining surgically implanted device that is implanted within a patient's body to help patient's that are diagnosed with late stage (CHF) Chronic Heart Failure. The LVAD and or RVAD must be supported by two external batteries and a controller, that monitors the heart. The drive line exits from its placement internally through an opening located in the abdomen, which feeds through an opening in the mid to lower abdominal areas. These support peripherals must be close to the body at all times.

The cosmetic cover will be offered in many different colors, materials/fabrics, styles and can be accessorized with additional embellishments “but, not limited to” such as pockets, zippers, buttons, buckles, handle(s) and tassels to add personalized detail and keep bags up to date with the current fashion trends.

The following invention; with its materials/fabrics and patterns; will be cut and sewn and assembled with a manufacturer, for retail sale.

Referring first to FIG. 1, according to a least one aspect of the invention, is shown in 1(A)medical device bag cover as it would appear after the 1(D) medical device bag which contains the LVAD/RVAD (not shown) has been inserted into 1(A) medical device bag cover.

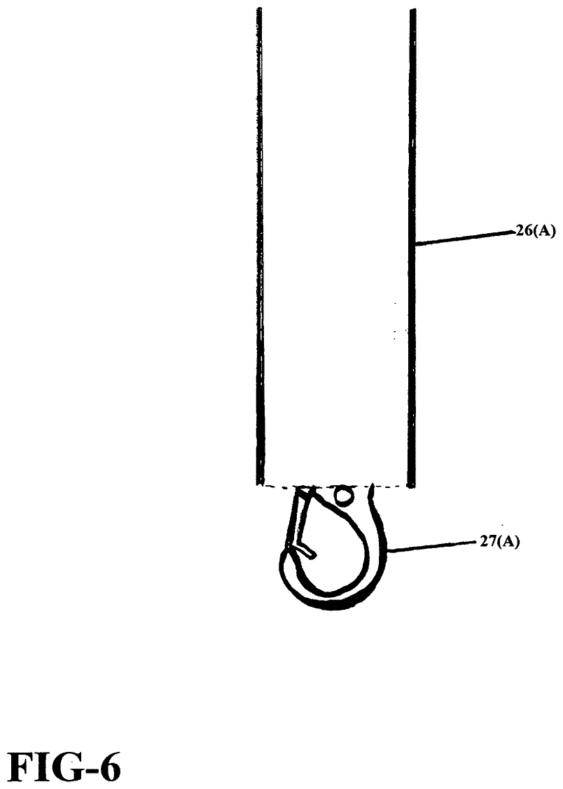

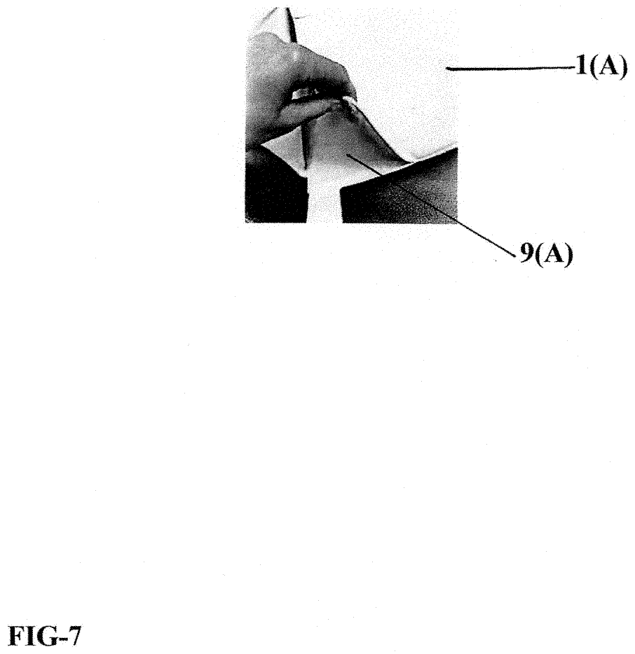

Medical device bag cover shown in FIG. 1 includes a 9(A) right-side opening/slit and/or 9(B) left side opening/slit panel; (which is not shown in FIG.1 drawings, and is listed just for references only); a clearer understanding of such an opening/slit can be seen in a finished prototype sample in FIG.7; this opening/slits, creates a passing for the exposed portion of the 11 driveline.; 8 shows how the driveline would appear after exiting the 1(A) medical device bag cover through 9(A) slit opening, and under the tab of 3(A) right tabs and 3(B) left tabs; where the tabs of 3(A) and 3(B) with its magnetic closure of 5 and 6 on both the right and left side(s) portion of the cover; tabs will be fully unfastened in order to accommodate driveline of 11 of the 1(D) medical device bag; when medical device bag; 1(D) has completely 25 assimilated in the medical bag cover of 1(A) the tabs of 3(A) and 3(B) will be joined with 5 and 6 magnetic front panel closures of the bag cover: the 2(A) interior flap of medical device bag cover will be pulled over the medical device bag and with the magnetic closures of 4 and 7; the flap and the front panel of the medical device bag cover; will be fasten together to give symmetry to the bag cover; with 26(A) medical device bag strap cover and 19 referring to the medical device bag strap with attached 27(B) “lobster clasp” which can be seen in FIG.6; will be reattached to 13 which refers to the fabric attached to medical device bags D rings 15(A)/15(B) and 14(A) 14(B) for securing the hardware in place of the 1(D) medical device bag and 19 the medical device bag strap will be fully integrated through the strap cover 26(A) opening; to create the illusion of a purse or messenger style bag.

as shown in the detailed drawing of FIG. 2; this will offer an exploded view of how all the pieces will be assimilated together.

Referring to all the pattern pieces of FIG. 2. comprising of 1(A), a three-dimensional medical device bag cover; with 26(A) medical device strap cover and 28(B) (Not Shown in Drawings, and for reference purposes only) fashionable removable shoulder pad ; these pieces will be included for creating; 28(A) a three-piece kit; with the pattern of; 1(B) front and 22 (A) back fabric panels, including a front and back panel of 1(C) cotton blend fabric for the internal lining; these pieces will be sewn by placing; “right sides together” an stitching from the middle portion of right and left side (s) of 1(B) and 22(A) medical device bag cover panels , to the bottom; 9(A) right-side (opening/slit) panel and/or 9(B)” not shown in FIG. 1 drawings and is listed for references only for left side opening/slit panel; this is created for passing the exposed portion of the 11 driveline.; 8 shows how the driveline would appear after exiting; 1(A) this will create a side panel; that ultimately will; create the opening(s)/slit (s) of 1(A) wherein; a 45-degree angle will be stitched from side(s) panels; 22(B) refers to the bottom of the medical device bag cover that will be created from the 1(B), front and 22(A) back panels; this creates a rectangle shaped bottom 22(B): the tabs of, 3(A) and 3(B) with its magnetic closure 5 and 6 on both the right and left side(s) portion of the cover will be fully unfastened in order to accommodate the 25 insertion of the 1(D) medical device bag and will be refastened by connecting tabs 3(A)/3(B) with front panel of the bag cover to close side (s); this will insure proper positioning and secure fastening after medical device bag is fully assimilated into the cover.

Medical device bag cover in FIG. 2 comprises of; 2(B) exterior flap of medical device bag cover; a right 3(A) right tabs and left 3(B)left tabs: these tabs; with 5(A) female magnetic portions of closure for tabs and 6 male magnetic portions of tabs; this will allow the bag to be opened and closed easily and securely.

FIG. 3 refers to the 22(A); the back panel of the medical device cover 23(A)Right and 23(B) left belt loops will be created after 1(B) front and 22(A) back panels that are stitched together. safety belt loops are added to the 22(A)-back panel of the bag cover to mimic the manufacturers recommended safety belt loops of the 1(D) HeartWare medical device bag. 24 demonstrate how the safety belt will be integrated though the 22(A)-back panel of, medical device bag cover and through the loops 23 (A) and 23(B).

In FIG. 3 a drawing depicting the partial driveline 8 exiting through opening/slit of 9(A)and under tab of 3(A) also showing in; FIG. 3 views of 19 the medical device bags strap; this drawing will give a visual of how it will appear after 1(A) medical device bag cover is has been placed and with its 2(B)-front flap closing.

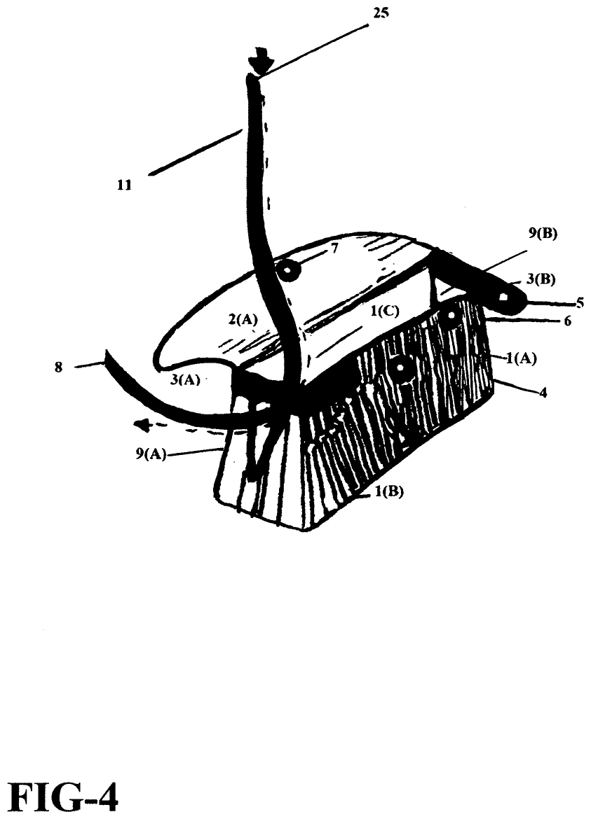

FIG. 4 the illustration of 25 refers to the direction that the driveline would be negotiated through the interior of the medical device bag cover and exiting through the opening/slit of 9(A) and under tab of 3(A); also shown in this view is 1(C) this is a reference to the internal fabric/material lining placed 1(A) in the medical bag device cover.

FIG. 5 is a view of the of the strap cover 26(A) that was modified to camouflage the 19 medical device safety straps; also, in this view a clearer understanding, of how the strap of the 1(A) medical device bag will be integrated through the medical device cover.

FIG. 6 is a view of 26(A) medical device safety strap cover, with its attaching straps connectors 27(A) lobster clasp, that will be attached to the D ring 15(A)/15(B) and 14(A)/14(B).

FIG. 7 refers to the 9(A) opening/slit of the medical device bag covers actual prototype. and a partial view of the front of the 1(A) medical device bag cover.

FIG. 8;—(C) refers to both the right and left (side(s) panel(s) of medical device bag cover, that is created by 1(B) and 22(A).

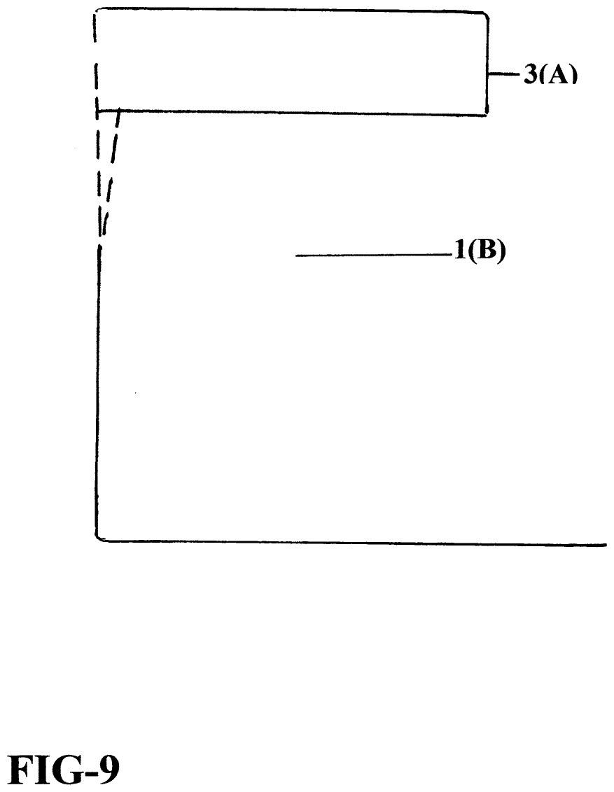

FIG. 9 is a view of the prototypes 3(A) tab as it would appear on 1(B) front panel of the medical device cover.

FIG. 10 refers to the bottom of the medical device bag cover 22(B); that will be created from 1(B) front panel of the medical bag cover and 22(A) back panel of the medical device cover.

FIG. 11 refers to the actual prototype bag 28(A); shown is the medical device bag covered 1(A) and covered strap 26A); the removable shoulder pad 28(B) (not shown in this picture) will also be included; these products will be sold as a three-piece kit.

The claimed invention is:

1. A fashionable cover, adapted to conceal the exterior components of LVADs and RVADs, also known as VADs, which are life-sustaining medical devices surgically implanted within the body to assist with cardiac function for patients diagnosed with late stage heart failure, the cover comprising a front panel ,back panel and a flap ,creates an open three-dimensional, generally rectangular-shaped compartment encompassing an opening of sufficient size to comfortably accommodate the HeartWare medical device bag; without altering the function of the device(s) as provided by the manufacturer, the bag covers will help patients conceal the exterior components of their LVAD/RVAD bag and strap, to create the illusion that the patient is carrying a purse or messenger-style bag. The cover(s) will be practical and functional for both men and women.

2. The cover of claim 1, comprising of an opened, three-dimensional cover compartment, including a front panel, back panel and flap; right and left belt loops will be created after front and back panels are stitched together, safety belt loops are sewn to the back panel of the bag cover, to mimic the manufactures recommended safety belt loops of the HeartWare medical device bag.

3. The cover of claim 1, comprising of a three-dimensional cover; with front and back fabric panels, including a front and back panel of a cotton blend fabric for the internal lining will be sewn and attached by placing; “right sides together” a stitching from the middle portion of right and left side(s) of bag cover, to the bottom side; this will create the side panel(s); the top portion from the middle of side(s) panel(s) to the upper part of front and back panels will remain opened; this will create the opening(s)/slit(s) in order to position the driveline safely through the opening (s)/slit(s) of the right and left side(s); the bottom will be created by sewing the bottom portion of front and back panel(s) closed, whereas; a 45 degree angle will be stitched from side(s) panel(s) to the bottom of front and back panel(s) ;this will create a rectangle shaped bottom where; the tabs, with its magnetic closures on both the right and left side(s) portion of the cover; will be fully unfastened in order to accommodate the insertion of the medical device bag , and will be refastened by connecting tabs, with front panel of the bag cover to close side(s); this will insure proper positioning and secure fastening after medical device bag is fully assimilated into the cover.

4. The cover of claim 2, comprises an external flap for opening and closing of the bag cover that will comprise of a male and female magnetic closure that will be amalgamated together.

5. The fashionable cover of claim 1, adapted to conceal the exterior of the HeartWare medical device bag detachable safety strap, will be covered in coordinated fabric to create the impression that the patient is carrying a purse or messenger-style bag.; the strap will be assimilated through the strap cover by way of two concealed opening in strap cover that will be hidden visually by a removable extra padded shoulder pad made in coordinated fabric used in the related bag cover.

6. The fashionable cover of claim 1, adapted to conceal the exterior of the HeartWare medical device bags safety strap; the safety strap will be negotiated through the strap cover with a flexible zip tie, that will have a small loop created on its end; this will be connected to the hardware of the medical strap (i.e. the lobster clasps) that is connected to the medical strap; and pulled gently and completely through the bag cover and attached back to the medical device bag without compromising the integrity of the medical bag.

7. The fashionable cover of claim 1; will be offered in many different colors, materials/fabrics, styles and can be accessorized with additional embellishments such as pockets, zippers, buttons, handle and tassels to add personalized detail and keep bags up to date with the current fashion trends.

8. The cover claims of 1-7; fashionable medical device bag cover, medical device bags safety strap cover and extra padded shoulder pad, will be sold as a three-piece kit.
